(19) United States
(12) Patent Application Publication
(10) Pub. No.: US 2010/0036327 A1
(43) Pub. Date: Feb. 11, 2010

(54) FLOW PREVENTION, REGULATION, AND SAFETY DEVICES AND RELATED METHODS

(75) Inventor: Paul Mario DiPerna, San Clemente, CA (US)

Correspondence Address: Luce, Forward, Hamilton & Scripps LLP, 2050 Main Street, Suite 600, Irvine, CA, 92614, US

(73) Assignee: TANDEM DIABETES CARE, INC.

(21) Appl. No.: 12/189,070

(22) Filed: Aug. 8, 2008

(51) Int. Cl. A61M 5/00

(52) U.S. Cl. 604/247

(57) ABSTRACT

A football-shaped blockage within a vessel lumen designed to prevent any flow material from passing through the vessel lumen when suction is applied below the blockage or when the flow material pressure is below a given value, and further designed to allow flow when the flow material pressure increases above the given value by allowing the vessel lumen walls to expand around the football-shaped blockage or, alternately, to allow the football-shaped blockage to collapse in response to the increased pressure. A series of compressible members contained within the vessel lumen may also be coupled with the above mentioned blockage device to limit the flow rate beyond the given value. The compressible members are designed to partially restrict flow so as to initially allow for only moderate increases in flow rate with respect to flow material pressure, but with a pressure above a second given material pressure, greater increases in flow rate are allowed.

### BACKGROUND OF THE DISCLOSURE

This disclosure relates to flow regulators designed to prevent flow through a vessel lumen when suction is applied or when the pressure is below a given value and further designed to ensure a predictable and stepped flow rate increase relative to a pressure increase above the given value.

### SUMMARY OF THE DISCLOSURE

A football-shaped blockage within a vessel lumen designed to prevent any flow material from passing through the vessel lumen when suction is applied below the blockage or when the flow material pressure is below a given value, and further designed to allow flow when the flow material pressure increases above the given value by allowing the vessel lumen walls to expand around the football-shaped blockage or, alternately, to allow the football-shaped blockage to collapse in response to the increased pressure. A series of compressible members contained within the vessel lumen may also be coupled with the above mentioned blockage device to limit the flow rate beyond the given value. The compressible members are designed to partially restrict flow so as to initially allow for only moderate increases in flow rate with respect to flow material pressure, but with a pressure above a second given material pressure, greater increases in flow rate are allowed.

According to a feature of the present disclosure, a flow regulation device is disclosed comprising a vessel lumen for transporting a flow material and a blockage member disposed within the vessel lumen and designed to prevent the flow material from flowing through the vessel lumen when suction is applied or when the flow material pressure is below a given value, wherein, in response to an increase in the flow material pressure above the given value, the vessel lumen is designed to sufficiently expand around the blockage member to create a channel through which flow material may flow.

According to another feature of the present disclosure, a method of restricting flow through a vessel lumen is disclosed comprised of providing a vessel having a lumen and a blockage member disposed along the flow path of a flow material and configured to prevent the flow material from flowing through the vessel when the flow material pressure falls below a predetermined value.

According to yet another feature of the present disclosure, a method of regulating flow through a vessel lumen is disclosed comprised of providing a vessel having a lumen, a blockage member disposed along the flow path of a flow material and configured to restrict the flow material as it flows through the vessel lumen when suction is applied or in the absence of a flow material pressure at or greater than a first threshold pressure, and a set of at least one compressible member disposed along the flow path of the flow material and configured to expand radially as the pressure of the flow material increases.

According to still another feature of the present disclosure, a flow regulation device is disclosed comprising a vessel lumen for transporting a flow material, a blockage member disposed along the flow path of a flow material and configured to restrict the flow material as it flows through the vessel lumen when suction is applied or in the absence of a flow material pressure at or greater than a given value, wherein, in response to an increase in the flow material pressure above the given value, the blockage is designed to sufficiently compress to create a channel through which flow material may flow.

### DRAWINGS OF THE DISCLOSURE

The above-mentioned features and objects of the present disclosure will become more apparent with reference to the following description taken in conjunction with the accompanying drawings wherein like reference numerals denote like elements and in which:

FIG. 1 is a cut-away view of an embodiment of a system of the flow prevention, regulation, and safety devices of the present disclosure within a lumen that is in communication with both a pump or flow material source and a patient or flow material destination;

FIG. 2 is a side sectional view of an embodiment of the flow prevention, regulation, and safety devices of the present disclosure disposed within a lumen with the flow material pressure in the no flow regime;

FIG. 3 is a side sectional view of an embodiment of the flow prevention, regulation, and safety devices of the present disclosure disposed within a lumen with the flow material pressure just above the no flow regime;

FIG. 4 is a graph of embodiments of the flow prevention, regulation, and safety devices of the present disclosure illustrating the behavior of the flow rate through the lumen with respect to flow material pressure;

FIG. 5A is a side sectional view of an embodiment of the flow prevention, regulation, and safety devices of the present disclosure disposed within a lumen with the flow material pressure in the no flow regime;

FIG. 5B is a top sectional view of an embodiment of the flow prevention, regulation, and safety devices of the present disclosure taken generally along line 5B-5B with the flow material pressure in the no flow regime;

FIG. 6A is a side sectional view of an embodiment of the flow prevention, regulation, and safety devices of the present disclosure disposed within a lumen with the flow material pressure in the slow flow regime;

FIG. 6B is a top sectional view of an embodiment of the flow prevention, regulation, and safety devices of the present disclosure taken generally along line 6B-6B with the flow material pressure in the slow flow regime;

FIG. 7A is a side sectional view of an embodiment of the flow prevention, regulation, and safety devices of the present disclosure disposed within a lumen with the flow material pressure in the fast flow regime;

FIG. 7B is a top sectional view of an embodiment of the flow prevention, regulation, and safety devices of the present disclosure taken generally along line 7B-7B with the flow material pressure in the fast flow regime; and

FIG. 8 is a graph of embodiments of the flow prevention, regulation, and safety device of the present disclosure illustrating the behavior of the flow rate through the lumen with respect to flow material pressure.

### DETAILED DESCRIPTION OF THE DISCLOSURE

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction (inclusive of the term “and”) and shall not indicate an exclusive disjunction unless expressly indicated as such or notated as “xor.”

As used in the present disclosure, the term “compress” or “compression” shall be defined as the decrease in cross-sectional area of the vessel lumen or a decrease in volume of the compressible members of the present disclosure.

As used in the present disclosure, the term “expand” or “expansion” shall be defined as the increase in cross-sectional area of the vessel lumen or an increase to the volume of the compressible members of the present disclosure.

Disclosed is a device for preventing flow material from passing though a lumen when suction is applied below the device or when the flow material pressure is below a threshold value, comprising a blockage within the lumen that is designed to have a larger diameter than the inner diameter of the vessel lumen. In the absence of a threshold pressure, the blockage prevents flow material from passing through the lumen when the flow material pressure is below a threshold pressure. The device is further designed to allow the walls of the vessel lumen to increase in diameter in response to an increase in flow material pressure above the given value or, alternately, the blockage may compress in response to an increase in flow material pressure. The disclosed device may further comprise compressible members to control the rate at which the flow of flow material increases with respect to the pressure of the flow material. The compressible pieces further provide for any number of stepped rates of increase of flow rate with respect to flow material pressure. The compressible pieces are designed to create pressure regions wherein any increase in flow material pressure results in an increase in flow rate, each region having its own specific flow rate profile.

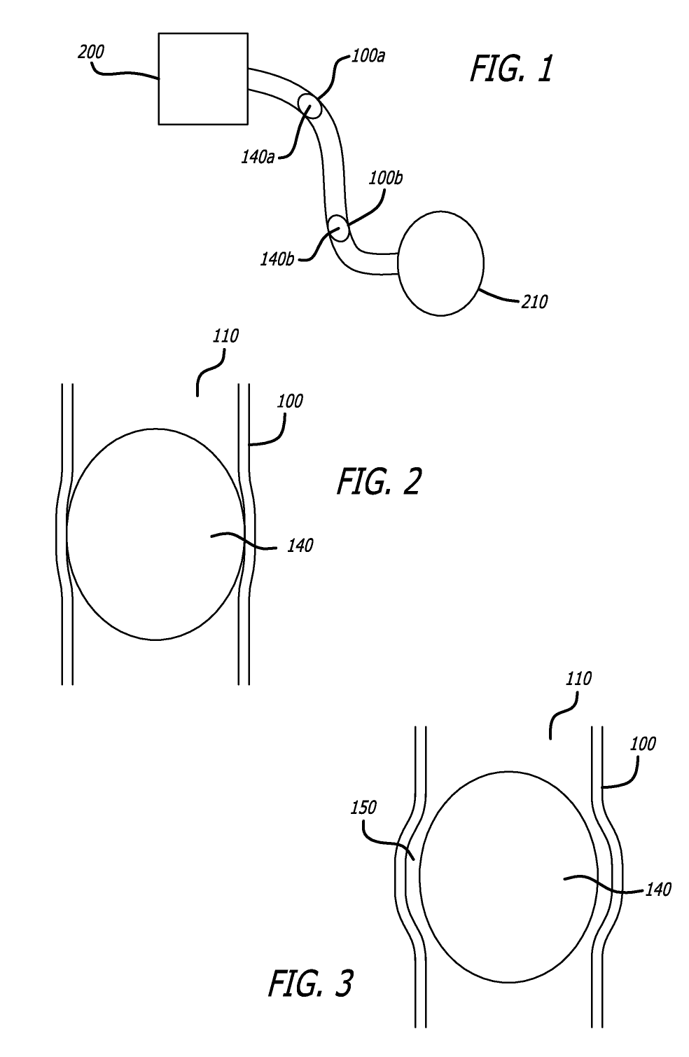

According to embodiments of the present disclosure and as illustrated in FIG. 1, a flow regulation device is illustrated in communication with a pump or flow material source and a patient or flow material destination. Pump 200 is the source a flow material that flows through vessel lumen 110 to patient 210. FIG. 2 illustrates flow regulation device 100 comprising, according to embodiments, vessel lumen 110 and blockage 140. Vessel lumen 110 transports a flow material from pump 200 to patient 210 and contains blockage 140, which may be shaped like a cylinder with rounded ends, somewhat similar to a football. Flow regulation device 100 may be positioned anywhere within vessel lumen 110. As illustrated in FIG. 1, flow regulation device 100A may be positioned closer to pump 200, or flow regulation device 100B may be positioned closer to patient 210.

FIG. 2 further illustrates that blockage 140 is designed to have a diameter slightly larger than the inner diameter of vessel lumen 110, such that the walls of vessel lumen 110 must expand slightly to accommodate blockage 110. The result is that no flow material may pass through vessel lumen 110 when the pressure of the flow material is below a predetermined pressure, termed a cracking point. A further result of the design of flow regulation device 100 is that even suction or a negative pressure below blockage 140 will not cause any flow material to flow passed blockage 140. Any negative pressure in vessel lumen 110 below blockage 140 will only tend to tighten the walls of vessel lumen 110 around blockage 140.

Despite the snug fit of blockage 140 within vessel lumen 110, blockage 140 must be secured within vessel lumen 110 such that an expansion of the walls of vessel lumen 110 does not allow blockage 140 to be pushed downstream by the flow material. According to embodiments, blockage 140 may be secured within vessel lumen 110 by connecting blockage 140 via a tether to pump 200, the tether being secured, according to embodiments, to pump 200 at a point at which pump 200 communicates with vessel lumen 110.

The utility of the above mentioned design features is illustrated in FIG. 1. As mentioned, a purpose of flow regulation device 100 is to prevent all flow material from passing through vessel lumen 110 when the flow material pressure is below the cracking point. Ideally, if it is desired to prevent all flow of flow material through vessel lumen 110 to patient 210, pump 200 is merely turned off or prevented from allowing any more flow material from entering vessel lumen 110. Unfortunately, even preventing additional flow material from entering vessel lumen 110 does not eliminate the already existing flow material pressure within vessel lumen 110. The mere presence of flow material in vessel lumen 110 creates a hydraulic head equal to the height difference between pump 200 and flow regulation device 100B. Flow regulation device 100B is designed to withstand such a hydraulic head and prevent any flow material from flowing passed blockage 140B to patient 210. To prevent flow in such a situation, flow regulation device 100B is designed to have a cracking point equal to or greater than the hydraulic head.

Alternatively, flow regulation device 100A may be positioned near pump 200. If it is desired to cease all flow through vessel lumen 110, pump 200 is shut off so as to prevent any additional flow material from entering vessel lumen 110. In this situation, though, the flow material already in vessel lumen 110 will produce a negative pressure below blockage 140A. As discussed previously, any negative pressure below blockage 140 will only tend to tighten the walls of vessel lumen 110 around blockage 140A, thus preventing the flow of flow material to patient 210.

FIG. 3 illustrates the result of increasing the flow material pressure above the cracking point such that the walls of vessel lumen 110 expand around blockage 140 to create channel 150 through which flow material may pass. Further increasing the flow material pressure causes further expansion of the walls of vessel lumen 110 and an increase in the flow rate of the flow material.

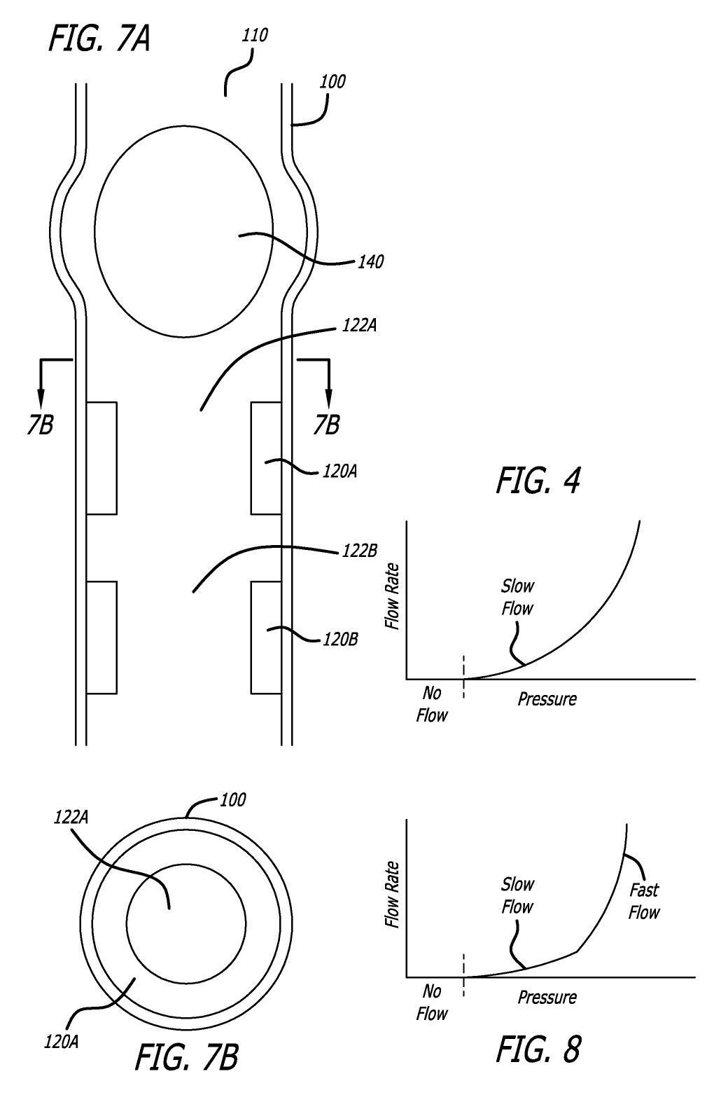

FIG. 4 illustrates the flow rate behavior of flow material through flow regulation device with respect to the flow material pressure. Below the cracking point, blockage 140 prevents any flow material from passing through vessel lumen 110. This is the no flow regime as illustrated in FIG. 4. Increasing the flow material pressure above the cracking point causes the walls of vessel lumen 110 to expand and allow flow. Further increasing the flow material pressure results in an exponentially increasing flow rate of flow material through flow regulation device 100. This is achieved as channel 150 continues to increase in diameter around blockage 140 in response to the increasing flow material pressure.

According to additional embodiments of the present disclosure, blockage 140 may be constructed of a compressible material such that an increase in flow material pressure compresses blockage 140 reducing its cross-sectional area, thus creating channel 150 and allowing flow material to pass through flow regulation device 100. According to embodiments, both blockage 140 and vessel lumen 110 may be constructed of non-rigid materials, though flow regulation device 100 would still perform as desired if vessel lumen 110 were rigid and blockage 140 were compressible. It should be remembered that all of the advantages of flow regulation device 100 outlined above would continue to apply to flow regulation device 100 when blockage 140 is designed to be compressible. For example, when suction or a negative pressure is applied behind blockage 140, the compressible material of blockage 140 will tend to enlarge and further obstruct the flow path of flow material through vessel lumen 110.

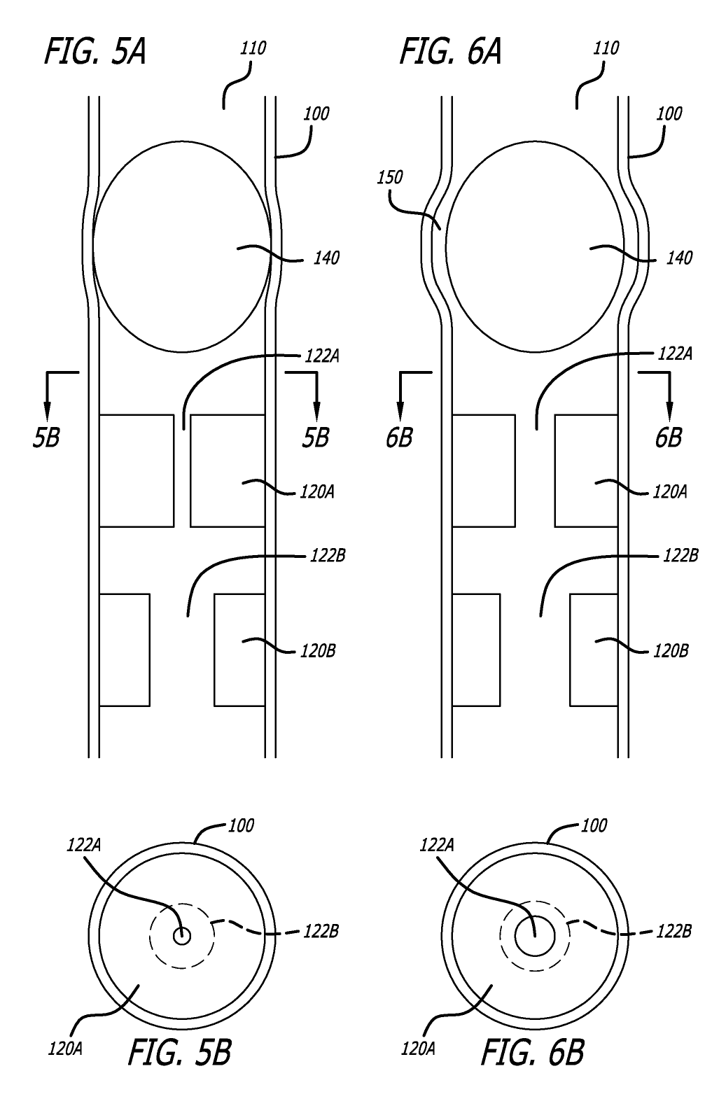

According to additional embodiments of the present disclosure and as illustrated in FIG. 5A, a flow regulation device is shown. Flow regulation device 100 comprises, according to embodiments, vessel lumen 110 which transports a flow material and contains, in addition to blockage 140, a plurality of compressible members 120A and 120B. Compressible members 120A and 120B are positioned to be below blockage 140. If desired, additional compressible members may be disposed with vessel lumen 110 either before or after blockage 140.

All compressible members 120A, 120B, etc. are composed of a compressible material such as an elastomer whereby they compress in response to an increase in flow material pressure of flow material in vessel lumen 110. As known and understood by artisans, each compressible member 120A, 120B, etc. may be made from the same or different elastomeric materials and have the same or different compression profiles, according to embodiments.

According to embodiments, blockage 140 is not used as part of flow regulation device 100. Accordingly, the flow profile (described below) starts at some baseline flow rate, which is modifiable solely with compressible members 120A and 120B, for example.

Also illustrated in FIGS. 5A and 5B, according to embodiments, is the relative size difference between compressible members 120A and 120B. Compressible member 120A is designed to restrict flow to a greater extent than compressible member 120B. This is accomplished by designing compressible member 120A to include channel 122A with a smaller diameter than corresponding channel 122B. As illustrated in FIG. 5A, channel 122B has a larger diameter than channel 122A.

As the flow material pressure increases above the first threshold pressure, or cracking point, according to embodiments, the wall of vessel lumen 110 expands around blockage 140 allowing flow material to flow through channel 150 and through vessel lumen 110. Alternately, according to embodiments, blockage 140 may be constructed of a compressible material so as to compress in response to a flow material pressure above the first threshold pressure. Compressible members 120A and 120B, which may be comprised of compressible rings attached to the inner wall of vessel lumen 110, are designed and configured to only partially restrict flow material at low flow material pressures. Alternatively, compressible members 120A and 120B may be attached to an outer wall of vessel lumen 110. As illustrated in both FIG. 6A and FIG. 6B, with an increase in pressure flow material begins to compress compressible member 120A. An increase in flow material pressure of the flow material further causes a compression of compressible member 120A and an enlargement of channel 122A.

FIG. 7A illustrates the result of a flow material pressure greater than a second threshold pressure. The increased flow material pressure causes compressible member 120A to fully compress allowing channel 122A to achieve its greatest diameter. Because channel 122B is designed with a larger initial diameter than channel 122A, the initial compression of compressible member 120A and increased flow of flow material may have relatively little effect on compressible member 120B. But with a flow material pressure at or above the second threshold pressure, compressible member 120A is compressed to a maximum, and any increase in flow material pressure causes compressible member 120B to compress and channel 122B to increase in diameter, thus allowing an even greater increase in the flow of flow material.

FIG. 7B illustrates the larger diameters of both channel 122A and 122B such that the two channels are relatively equal in diameter and flow material is permitted to flow through vessel lumen 110 with the least amount of restriction possible. According to embodiments, channels 122A and 122B may be configured to substantially reach their maximum size with substantially different diameters.

FIG. 8 illustrates the flow rate of flow material with respect to the flow material pressure. As illustrated, the flow rate of flow material is divided into three general regimes, no flow, slow flow, and fast flow, each regime generally controlled first by blockage 140 and subsequently by the compression of each successive compressible member contained in or on vessel lumen 110. Artisans will appreciate that the linearity illustrated in FIG. 3 is for illustration of the general principle only; in actual practice, the lines may be non-linear. The no flow regime corresponds to a flow material pressure below the first threshold pressure, in which blockage 140 restricts substantially all flow of flow material through vessel lumen 110. The slow flow regime corresponds to a flow material pressure above the first threshold pressure, or cracking point, but below the second threshold pressure, in which compressible member 120A greatly restricts the flow of flow material. Increasing the flow material pressure compresses compressible member 120A causing channel 122A to increase in diameter and allowing an increase in the flow rate. The fast flow regime corresponds to a flow material pressure above the second threshold pressure, in which compressible member 120B becomes the only compressible member to compress in response to an increase in flow material pressure. Increasing the flow material pressure compresses compressible member 120B causing channel 122B to increase in diameter and resulting in a faster increase in the flow rate with respect to a corresponding increase in flow material pressure.

According to embodiments, a method is disclosed whereby the flow material being transported from pump 200 through vessel lumen 110 to patient 210 is effectively stopped. Flow regulation device 100A may be connected near pump 200. Flow regulation device 100B may also be connected near patient 210. In either position, flow regulation device 100 effectively stops all movement of flow material through vessel lumen 110 when the flow material pressure falls below the cracking point.

According to embodiments, an additional method is disclosed whereby the flow rate of flow material through vessel lumen 110 is affected. Flow regulation device 100 is connected to pump 200 or a flow material source and to patient 210 or a flow material destination. Flow regulation device 100 is positioned with blockage 140 toward pump 200. If properly connected, flow material should flow from pump 200 and first contact blockage 140. With the connections established, flow material may flow from the flow material source to flow regulation device 100. When the flow material pressure is below the cracking point, essentially no flow material passes blockage 140. An increase in the flow material pressure expands the walls of vessel lumen 110 to increase in diameter around blockage 140. According to embodiments mentioned above, blockage 140 may comprise a compressible material such that an increase in flow material pressure causes a compression of blockage 140 within vessel lumen 110. Eventually the pressure of the flow material reaches the cracking point, or first threshold pressure. The first threshold pressure is reached when the walls of vessel lumen 110 sufficiently expand to create channel 150 allowing flow material to flow passed blockage 140 and through the flow regulation device. Alternately, the first threshold pressure may be the pressure at which blockage 140, if compressible, compresses to the point of creating channel 150 so that flow material may flow passed blockage 140. When flow material passes blockage 140, it then flows passed compressible member 120A and through channel 122A. Any additional increase in pressure of the flow material compresses compressible member 120A causing channel 122A to enlarge and allowing a greater volume of flow material through flow regulation device 100. The pressure may be increased to reach a second threshold pressure. The second threshold pressure is reached when compressible member 120A is compressed and any additional pressure increase compresses compressible member 120B causing channel 122B to enlarge and allowing even more flow material through flow regulation device 100.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A device comprising:
a vessel lumen for transporting a flow material; and
a blockage member disposed within the vessel lumen and designed to prevent the flow material from flowing through the vessel lumen when suction is applied or when the flow material pressure is below a given value, wherein, in response to an increase in the flow material pressure above the given value, the vessel lumen is designed to sufficiently expand around the blockage member to create a channel through which flow material may flow.

2. The device of claim 1, further comprising a set of at least one compressible member disposed along the flow path of the flow material and configured to expand radially as the pressure of the flow material increases.

3. The device of claim 2, wherein the set of compressible members are each configured to expand radially at variable rates.

4. The device of claim 2, wherein each subsequent compressible member along the flow path does not substantially expand until the immediately previous compressible member has substantially expanded.

5. The device of claim 2, wherein each of the set of compressible members have variable diameters.

6. The device of claim 2, wherein each of the compressible members have variable modulus of elasticity, whereby each of compressible members expand radially as a function of pressure at different rates.

7. The device of claim 6, wherein each of the set of compressible members have variable diameters.

8. The device of claim 2, wherein the set of at least one compressible member is disposed at least partially on an exterior surface of the vessel.

9. A method comprising:
providing a vessel having a lumen and a blockage member disposed along the flow path of a flow material and configured to prevent the flow material from flowing through the vessel when the flow material pressure falls below a predetermined value.

10. The method of claim 9, wherein the blockage member prevents the flow material from flowing through the vessel when the flow material exerts a negative pressure on the blockage member.

11. A method comprising:
providing a vessel having a lumen and a blockage member disposed along the flow path of a flow material and configured to restrict the flow material as it flows through the vessel lumen when suction is applied or in the absence of a flow material pressure at or greater than a first threshold pressure, and a set of at least one compressible member disposed along the flow path of the flow material and configured to expand radially as the pressure of the flow material increases.

12. The method of claim 11, wherein the set of compressible members are each configured to expand radially at variable rates.

13. The method of claim 12, wherein each subsequent compressible member along the flow path does not substantially expand until the immediately previous compressible member has substantially expanded.

14. The method of claim 12, wherein each of the set of compressible members have variable diameters.

15. The method of claim 12, wherein each of the compressible members have variable modulus of elasticity, whereby each of compressible members expand radially as a function of pressure at different rates.

16. The method of claim 15, wherein each of the set of second compressible members have variable diameters.

17. The method of claim 12, wherein the set of at least one second compressible member is disposed at least partially on an exterior surface of the vessel.

18. A device comprising:
a vessel lumen for transporting a flow material; and
a blockage member disposed along the flow path of a flow material and configured to restrict the flow material as it flows through the vessel lumen when suction is applied or in the absence of a flow material pressure at or greater than a given value, wherein, in response to an increase in the flow material pressure above the given value, the blockage is designed to sufficiently compress to create a channel through which flow material may flow.

19. The device of claim 18, further comprising a set of at least one compressible member disposed along the flow path of the flow material and configured to expand radially as the pressure of the flow material increases.

20. The device of claim 19, wherein the set of compressible members are each configured to expand radially at variable rates.
